(19) United States
(12) Patent Application Publication
(10) Pub. No.: US 2014/0343290 A1
(43) Pub. Date: Nov. 20, 2014

(54) PROCESS FOR THE PREPARATION OF ATAZANAVIR OR ITS BISULFATE SALT

(71) Applicants: Rakesh Kumar Singh, Jaunpur (IN); Nagaraju Gottumukkala, West Godavari (IN); Mahavir Singh Khanna, New Delhi (IN); Rajesh Kumar Thaper, Jammu (IN); Mohan Prasad, Gurgaon (IN); Sudershan Kumar Arora, Gurgaon (IN)

(72) Inventors: Rakesh Kumar Singh, Jaunpur (IN); Nagaraju Gottumukkala, West Godavari (IN); Mahavir Singh Khanna, New Delhi (IN); Rajesh Kumar Thaper, Jammu (IN); Mohan Prasad, Gurgaon (IN); Sudershan Kumar Arora, Gurgaon (IN)

(21) Appl. No.: 14/235,127

(22) PCT Filed: Jul. 25, 2012
(86) PCT No.: PCT/IB2012/053805

§ 371 Date: Jul. 30, 2014

(30) Foreign Application Priority Data
Jul. 27, 2011 (IN) 2135/DEL/2011

(51) Int. Cl. C07D 213/42

(52) U.S. Cl. CPC C07D 213/42
USPC 546/332

(57) ABSTRACT

The present invention relates to an improved process for the preparation of atazanavir bisulfate, an inhibitor of retroviral aspartate protease. The process of the present invention comprises conversion of 1,1-dimethylethyl[(2S,3R)-4-chloro-3-hydroxy-phenylbutan-2-yl]carbamate (Formula II) into 1-[4-(pyridine-2-yl)-phenyl]-4(S)-5 hydroxy-2-N-tert-butoxycarbonylamino-5(S)—N—(N-methoxycarbonyl-(L)-tert-leucyl)amino-6-phenyl-2-azahexane (Formula VII) without isolating intermediate compounds formed therein, followed by its subsequent conversion to atazanavir or its bisulfate salt.

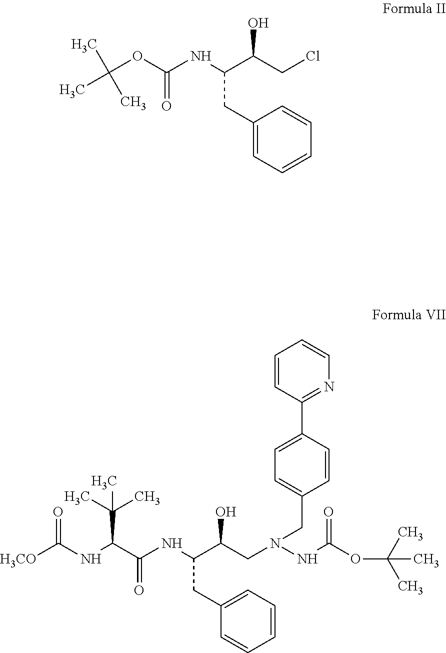 [SMILES: CC(C)(C)OC(=O)N[C@@H](Cc1ccccc1)[C@@H](O)CCl ; COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

### FIELD OF THE INVENTION

The present invention relates to an improved process for the preparation of atazanavir bisulfate, an inhibitor of retroviral aspartate protease. The process of the present invention comprises conversion of 1,1-dimethylethyl[(2S,3R)-4-chloro-3-hydroxy-1-phenylbutan-2-yl]carbamate (Formula II) into 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII) without isolating intermediate compounds formed therein, followed by its subsequent conversion to atazanavir.

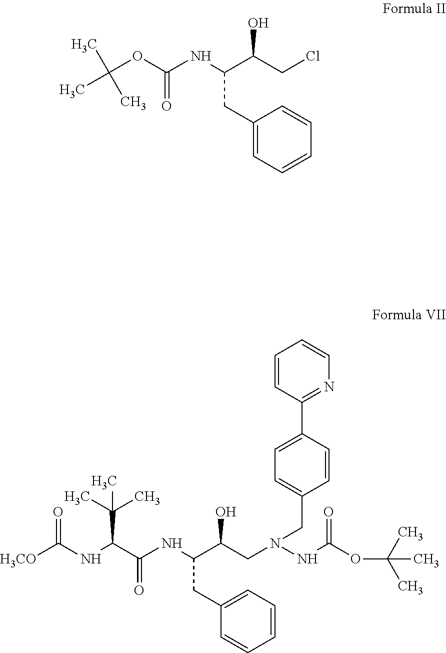 [SMILES: CC(C)(C)OC(=O)N[C@@H](Cc1ccccc1)[C@@H](O)CCl ; COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

### BACKGROUND OF THE INVENTION

Atazanavir and its bisulfate salt (1:1) are disclosed in U.S. Pat. Nos. 5,849,911 and 6,087,383, respectively. Atazanavir bisulfate is chemically known as (3S,8S,9S,12S)-3,12-bis(1,1-dimethylethyl)-8-hydroxy-4,11-dioxo-9-(phenylmethyl)-6-[[4-(2-pyridinyl)phenyl]methyl]-2,5,6,10,13-pentaazatetradecanedioic acid dimethyl ester, sulfate (1:1) and is represented by Formula I.

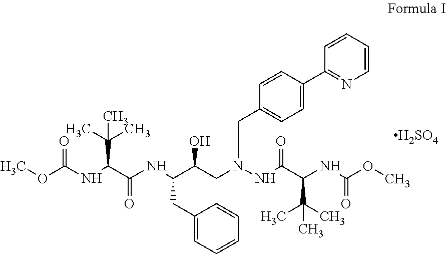

Atazanavir bisulfate is an inhibitor of retroviral aspartate protease and also known to have a high degree of inhibitory activity against the HIV virus.

U.S. Pat. Nos. 5,849,911; 6,300,519; and 6,110,946 describe a multistep process for the preparation of atazanavir based on different synthetic approaches and intermediates. One of the interesting approaches exemplified is condensation of either 1-[4-(pyridine-2-yl)-phenyl]-4(S)-hydroxy-2-amino-5(S)—N—(N-methoxycarbonyl-(L)-tert-leucyl)amino-6-phenyl-2-azahexane (Formula VIII) or 1-[4-(pyridine-2-yl)-phenyl]-4(S)-hydroxy-5(S)-2,5-diamino-6-phenyl-2-azahexane (Formula VIIIa)

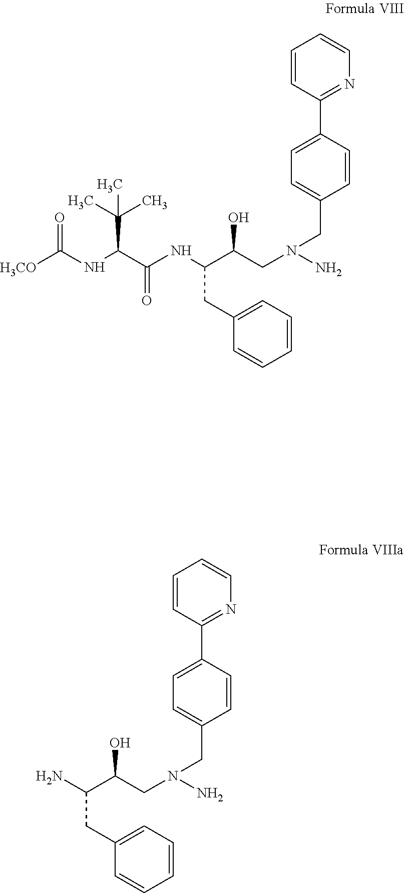 [SMILES: COC(=O)N[C@H](C(=O)N[C@@H](Cc1ccccc1)[C@@H](O)CN(N)Cc1ccc(-c2ccccn2)cc1)C(C)(C)C ; N[C@@H](Cc1ccccc1)[C@@H](O)CN(N)Cc1ccc(-c2ccccn2)cc1]

with N-methoxycarbonyl-(L)-tert-leucine in presence of TPTU [O-(1,2-dihydro-2-oxo-1-pyridyl)-N,N,N′,N′-tetramethyluroniumtetrafluoroborate] and Hunig base (N-ethyldiisopropylamine) to obtain atazanavir. Interestingly, most of the key intermediate compounds are either isolated or crystallized to get atazanavir.

U.S. Pat. Nos. 7,829,720 and 7,838,678 describe a process for the preparation of atazanavir comprising in-situ condensing 1-[4-(pyridine-2-yl)-phenyl]-4(S)-hydroxy-5(S)-2,5-diamino-6-phenyl-2-azahexane (Formula VIIIa) or its salt with excessive moles of 1-hydroxy-benzotriazole ester of N-methoxycarbonyl-(L)-tert-leucine in presence of K₂HPO₄.

PCT Publication No. WO 2008/065490 describes a multistep process for the preparation of atazanavir comprising condensing 1-[4-(pyridine-2-yl)-phenyl]-4(S)-hydroxy-5(S)-amino-2-N—(N-benzyloxycarbonyl-(L)-tert-leucyl)amino-6-phenyl-2-azahexane or a salt thereof with N-methoxycarbonyl-(L)-tert-leucine followed by subsequent replacement of benzyloxycarbonyl by methoxycarbonyl moiety.

PCT Publication No. WO 2010/146119 describes a process for preparing atazanavir or its pharmaceutically acceptable salts or solvate thereof comprising condensing 1-[4-(pyridine-2-yl)-phenyl]-4(S)-hydroxy-5(S)-2,5-diamino-6-phenyl-2-azahexane (Formula VIIIa) with N-methoxycarbonyl-(L)-tert-leucine using carbodiimide in the absence of 1-hydroxy-benzotriazole.

PCT Publication Nos. WO 2009/130534 and WO 2010/149356 also describe a process for preparing atazanavir or a salt thereof which are herein incorporated for reference.

There is still a need for an improved and simplified process for the preparation of atazanavir or salts thereof, which can reduce the burden of isolating, crystallizing, and purifying the intermediate compound(s), and thus minimize production time, provide high yield and is convenient to operate on a commercial scale. The present invention is associated with these advantages and enables practical and efficient manufacture of atazanavir or its salts. Added advantages of the present invention are cost effectiveness, readily accessible raw materials, less hazardous reaction conditions, and simple work up procedures which all make the process more robust and cost-efficient.

### SUMMARY OF THE INVENTION

The present invention provides a process for the preparation of atazanavir that comprises conversion of 1,1-dimethylethyl[(2S,3R)-4-chloro-3-hydroxy-1-phenylbutan-2-yl]carbamate (Formula II) into 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII) without isolating intermediate compounds formed therein, followed by its conversion to atazanavir.

In other words, the present invention provides a process for the preparation of atazanavir wherein only one intermediate compound of Formula VII is isolated during the entire process of manufacture.

The present invention also provides processes for the preparation of various intermediate compounds involved in the process of manufacture for atazanavir.

### DETAILED DESCRIPTION OF THE INVENTION

The term “atazanavir bisulfate”, as employed herein, refers to atazanavir bisulfate as well as atazanavir sulfate. The term “ambient temperature”, as employed herein, refers to a temperature range of 20° C. to 35° C.

The present invention can be explained by way of following the aspects.

A first aspect of the present invention provides a process for the preparation of atazanavir or its bisulfate salt comprising:
a) converting 1,1-dimethylethyl[(2S,3R)-4-chloro-3-hydroxy-1-phenylbutan-2-yl]carbamate (Formula II) into 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII)

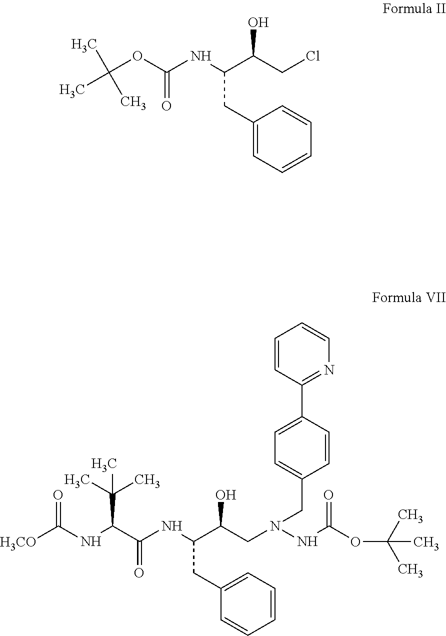 [SMILES: CC(C)(C)OC(=O)N[C@@H](Cc1ccccc1)[C@@H](O)CCl ; COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

without isolating any intermediate compound(s) involved in the conversion; and
b) converting the compound of Formula VII of step a) into atazanavir or its bisulfate salt.

In an embodiment of this aspect, the 1,1-dimethylethyl[(2S,3R)-4-chloro-3-hydroxy-1-phenylbutan-2-yl]carbamate (Formula II) is converted into 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII) without isolating (2S,3R)-2-amino-4-chloro-1-phenylbutan-3-ol (Formula III), methyl[(2S)-1-{[(2S,3R)-4-chloro-3-hydroxy-1-phenylbutan-2-yl]amino}-3,3-dimethyl-1-oxobutan-2-yl]carbamate (Formula IV) and/or methyl[(2S)-3,3-dimethyl-1-({ (1S)-1-[(2R)-oxiran-2-yl]-2-phenylethyl}amino)-1-oxobutan-2-yl]carbamate (Formula V) intermediate compounds.

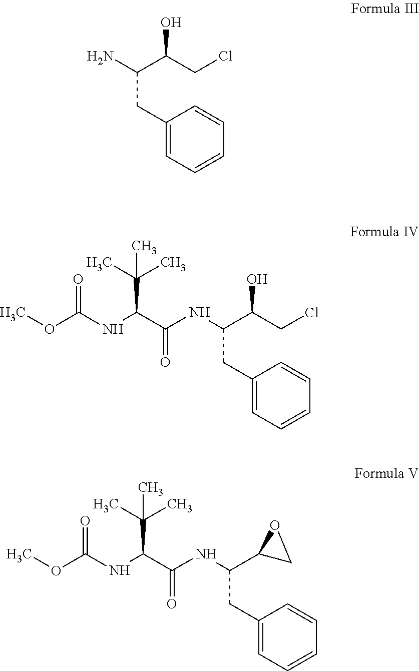

In another embodiment of this aspect, the 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII) is deprotected to obtain 1-[4-(pyridine-2-yl)-phenyl]-4(S)-hydroxy-2-amino-5(S)—N—(N-methoxycarbonyl-(L)-tert-leucyl)amino-6-phenyl-2-azahexane (Formula VIII) or a salt thereof, which upon treatment with an active ester of N-methoxycarbonyl-(L)-tert-leucine provides atazanavir.

In another embodiment of this aspect, atazanavir can be converted into its bisulfate salt by treating atazanavir base with sulfuric acid.

Accordingly, the 1,1-dimethylethyl[(2S,3R)-4-chloro-3-hydroxy-1-phenylbutan-2-yl]carbamate (Formula II) is deprotected followed by treatment with an active ester of N-methoxycarbonyl-(L)-tert-leucine to produce an intermediate compound of Formula IV, which is converted in-situ into the corresponding epoxy compound of Formula V. The epoxy compound is condensed in-situ with N-1-(tert-butoxycarbonyl)-N-2-[4-(pyridine-2-yl)benzyl]hydrazine of Formula VI

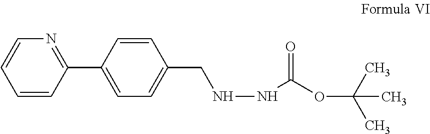 [SMILES: CC(C)(C)OC(=O)NNCc1ccc(-c2ccccn2)cc1]

to provide 1-[4-(pyridine-2-yl)-phenyl]-4(S)-hydroxy-2-N-tert-butoxycarbonyl amino-5(S)—N—(N-methoxycarbonyl-(L)-tert-leucyl)amino-6-phenyl-2-azahexane (Formula VII), which is finally isolated. This compound of Formula VII is deprotected and subsequently converted into atazanavir by treating with an active ester of N-methoxycarbonyl-(L)-tert-leucine in the presence of thionyl chloride or oxalyl chloride. The atazanavir so produced is then converted into its bisulfate salt (1:1).

A second aspect of the present invention provides a process for the preparation of atazanavir or its bisulfate salt comprising:
a) converting 1,1-dimethylethyl[(2S,3R)-4-chloro-3-hydroxy-1-phenylbutan-2-yl]carbamate (Formula II) into 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII)

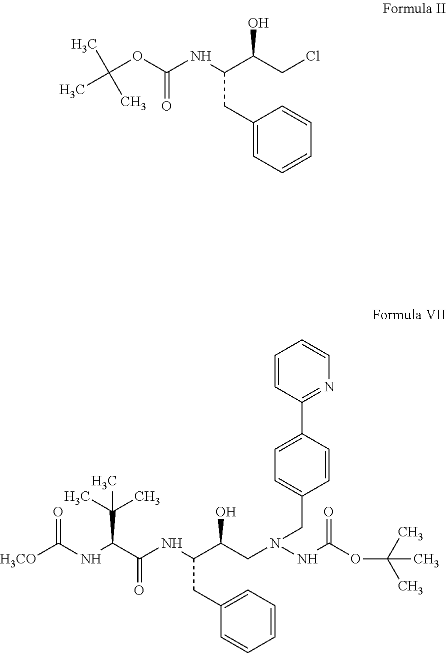 [SMILES: CC(C)(C)OC(=O)N[C@@H](Cc1ccccc1)[C@@H](O)CCl ; COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

without isolating intermediate compound(s) involved in the conversion, such as (2S,3R)-2-amino-4-chloro-1-phenylbutan-3-ol (Formula III), methyl[(2S)-1-{[(2S,3R)-4-chloro-3-hydroxy-1-phenylbutan-2-yl]amino}-3,3-dimethyl-1-oxobutan-2-yl]carbamate (Formula IV) or methyl[(2S)-3,3-dimethyl-1-({(1S)-1-[(2R)-oxiran-2-yl]-2-phenylethyl}amino)-1-oxobutan-2-yl]carbamate (Formula V); and

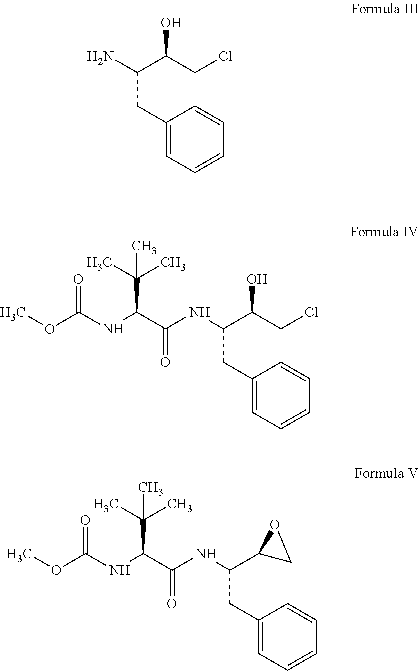

b) converting the compound of Formula VII of step a) into atazanavir or its bisulfate salt.

In another embodiment of this aspect, the 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII) is deprotected to obtain 1-[4-(pyridine-2-yl)-phenyl]-4(S)-hydroxy-2-amino-5(S)—N—(N-methoxycarbonyl-(L)-tert-leucyl)amino-6-phenyl-2-azahexane (Formula VIII) or a salt thereof, which on treatment with an active ester of N-methoxycarbonyl-(L)-tert-leucine provides atazanavir.

In another embodiment of this aspect, atazanavir can be converted into its bisulfate salt by treating atazanavir base with sulfuric acid.

Accordingly, 1,1-dimethylethyl[(2S,3R)-4-chloro-3-hydroxy-1-phenylbutan-2-yl]carbamate (Formula II) is deprotected followed by treatment with an active ester of N-methoxycarbonyl-(L)-tert-leucine to produce an intermediate compound of Formula IV, which is converted in-situ into the corresponding epoxy compound of Formula V. The epoxy compound is then condensed in-situ with N-1-(tert-butoxycarbonyl)-N-2-[4-(pyridine-2-yl)benzyl]hydrazine of Formula VI to provide 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII), which is isolated. This compound of Formula VII is deprotected and subsequently converted into atazanavir by treating with an active ester of N-methoxycarbonyl-(L)-tert-leucine in the presence of thionyl chloride or oxalyl chloride. The atazanavir so produced is then converted into its bisulfate salt (1:1).

A third aspect of the present invention provides a process for the preparation of atazanavir or its bisulfate salt by treating a reaction mixture comprising 1-[4-(pyridine-2-yl)-phenyl]-4(S)-hydroxy-2-amino-5 (S)—N—(N-methoxycarbonyl-(L)-tert-leucyl)amino-6-phenyl-2-azahexane (Formula VIII)

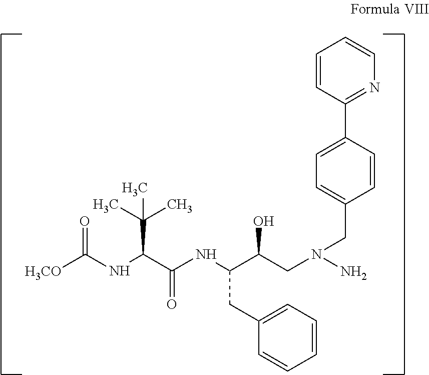

with an active ester of N-methoxycarbonyl-(L)-tert-leucine.

In an embodiment of this aspect, the treatment of the reaction mixture containing a compound of Formula VIII with an active ester of N-methoxycarbonyl-(L)-tert-leucine is carried out in the presence of thionyl chloride or oxalyl chloride.

In another embodiment of this aspect, reaction of the reaction mixture with an active ester of N-methoxycarbonyl-(L)-tert-leucine can be performed in the presence of a base and organic solvent.

The “base” can be selected from the group comprising hydroxides, carbonates or phosphates of alkali or alkaline earth metals. It can also be an organic base or a mixture of organic and inorganic bases. Some non-limiting examples of the base are sodium hydroxide [NaOH], potassium hydroxide [KOH], magnesium hydroxide [Mg(OH)₂], dipotassium hydrogen orthophosphate [K₂HPO₄], magnesium carbonate [MgCO₃], sodium carbonate [Na₂CO₃], potassium carbonate [K₂CO₃], pyridine, trimethylamine, triethylamine, diisopropylethylamine and/or N-methyl morpholine.

The “organic solvent”, as used herein, can be a polar solvent or a non-polar solvent selected from the group comprising methylene chloride, ethyl acetate, butyl acetate, dichloroethane, tetrahydrofuran, acetonitrile, acetone, dimethylsulfoxide, N,N-dimethylformamide, benzene, toluene, 1,4-dioxane, chloroform and/or diethyl ether.

In another embodiment of this aspect, N-methoxycarbonyl-(L)-tert-leucine is converted into its active ester by reacting it with a coupling agent selected from the group comprising O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide, and N-ethyl-N′-dimethylaminopropylcarbodiimide (EDC).

In another embodiment of this aspect, the active ester of N-methoxycarbonyl-(L)-tert-leucine is prepared by reacting it with 1-hydroxybenzotriazole (HOBT).

In another embodiment of this aspect, 1-[4-(pyridine-2-yl)-phenyl]-4(S)-hydroxy-2-amino-5(S)—N—(N-methoxycarbonyl-(L)-tert-leucyl)amino-6-phenyl-2-azahexane (Formula VIII) can be a salt of hydrochloric acid, hydrobromic acid, hydrofluoric acid, sulfuric acid, phosphoric acid, methanesulphonic acid or trifluoroacetic acid in the reaction mixture.

In another embodiment of this aspect, atazanavir can be converted into its bisulfate salt by treating it with sulfuric acid.

Accordingly, a reaction mixture containing 1-[4-(pyridine-2-yl)-phenyl]-4(S)-hydroxy-2-amino-5(S)—N—(N-methoxycarbonyl-(L)-tert-leucyl)amino-6-phenyl-2-azahexane (Formula VIII) can be obtained by following step a of the first aspect of the present invention to get 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane of Formula VII and then treating it with hydrochloric acid in dichloromethane/water solvent system followed by the separation of layers to collect the aqueous layer. This reaction mixture (aqueous layer) is then treated with an active ester of N-methoxycarbonyl-(L)-tert-leucine in the presence of thionyl chloride or oxalyl chloride, a base and organic solvent to obtain atazanavir, which is then converted into atazanavir bisulfate.

A fourth aspect of the present invention provides a process for the preparation of atazanavir or its bisulfate salt comprising the step of reacting (2S,3R)-2-amino-4-chloro-1-phenylbutan-3-ol represented by Formula III or a salt thereof

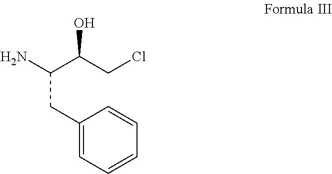

with an active ester of N-methoxycarbonyl-(L)-tert-leucine in the presence of thionyl chloride or oxalyl chloride to get methyl[(2S)-1-{[(2S,3R)-4-chloro-3-hydroxy-1-phenylbutan-2-yl]amino}-3,3-dimethyl-1-oxobutan-2-yl]carbamate of Formula IV.

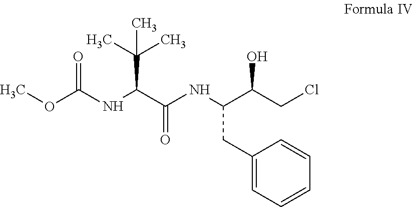

In an embodiment of this aspect, reaction of the compound of Formula III or a salt thereof with an active ester of N-methoxycarbonyl-(L)-tert-leucine can be performed in the presence of a base and an organic solvent.

The term “base”, as used herein, has the same meaning as defined in the previous aspect.

The term “organic solvent”, as used herein, has the same meaning as defined in the previous aspect.

N-methoxycarbonyl-(L)-tert-leucine can be converted into its active ester by reacting with a coupling agent selected from the likes of O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide, N-ethyl-N′-dimethylaminopropylcarbodiimide (EDC), etc.

In an embodiment of this aspect, the active ester of N-methoxycarbonyl-(L)-tert-leucine can be prepared by reacting it with 1-hydroxybenzotriazole (HOBT).

In another embodiment of this aspect, a “salt” of Formula III can be a salt of an acid selected from hydrochloric acid, hydrobromic acid, hydrofluoric acid, sulfuric acid, phosphoric acid, methane sulphonic acid, trifluoroacetic acid, or mixtures thereof.

To prepare atazanavir or its bisulfate salt, methyl[(2S)-1-{[(2S,3R)-4-chloro-3-hydroxy-1-phenylbutan-2-yl]amino}-3,3-dimethyl-1-oxobutan-2-yl]carbamate of Formula IV is converted into the corresponding epoxy compound of Formula V. The epoxy compound is then treated in-situ with N-1-(tert-butoxycarbonyl)-N-2-[4-(pyridine-2-yl)benzyl]hydrazine of Formula VI to provide 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII). This compound is then deprotected and subsequently converted into atazanavir by treating with an active ester of N-methoxycarbonyl-(L)-tert-leucine in the presence of thionyl chloride or oxalyl chloride. The atazanavir so produced is then converted into its bisulfate salt (1:1).

A fifth aspect of the present invention provides a process for the preparation of atazanavir or its bisulfate salt, which comprises the step of condensing methyl[(2S)-3,3-dimethyl-1-({(1S)-1-[(2R)-oxiran-2-yl]-2-phenylethyl}amino)-1-oxobutan-2-yl]carbamate (Formula V)

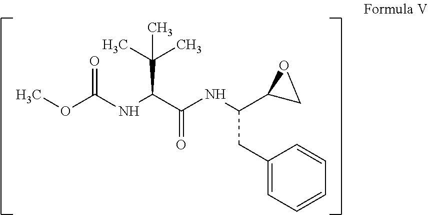

with N-1-(tert-butoxycarbonyl)-N-2-[4-(pyridine-2-yl)benzyl]hydrazine of Formula VI in-situ

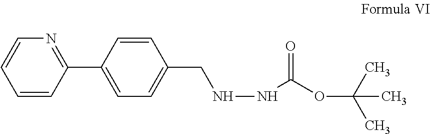 [SMILES: CC(C)(C)OC(=O)NNCc1ccc(-c2ccccn2)cc1]

to obtain 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII).

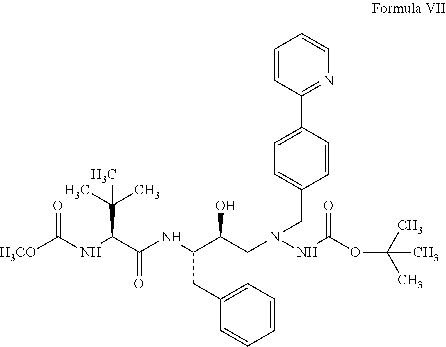 [SMILES: COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

Accordingly, the reaction mixture comprising methyl[(2S)-3,3-dimethyl-1-({(1S)-1-[(2R)-oxiran-2-yl]-2-phenylethyl}amino)-1-oxobutan-2-yl]carbamate (Formula V) is obtained by deprotecting 1,1-dimethylethyl[(2S,3R)-4-chloro-3-hydroxy-1-phenylbutan-2-yl]carbamate (Formula II) followed by treatment with active ester of N-methoxycarbonyl-(L)-tert-leucine to produce methyl[(2S)-1-{[(2S,3R)-4-chloro-3-hydroxy-1-phenylbutan-2-yl]amino}-3,3-dimethyl-1-oxobutan-2-yl]carbamate (Formula IV) which is converted in-situ into the corresponding “epoxy compound” of Formula V in the presence of N-1-(tert-butoxycarbonyl)-N-2-[4-(pyridine-2-yl)benzyl]hydrazine of Formula VI. The reaction mixture so obtained, comprising compound of Formula V, is then condensed in-situ with N-1-(tert-butoxycarbonyl)-N-2-[4-(pyridine-2-yl)benzyl]hydrazine of Formula VI to provide 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII). The compound of Formula VII is then converted into atazanavir or its bisulfate salt via the preparation of 1-[4-(pyridine-2-yl)-phenyl]-4(S)-hydroxy-2-amino-5(S)—N—(N-methoxycarbonyl-(L)-tert-leucyl)amino-6-phenyl-2-azahexane (Formula VIII).

The active ester of N-methoxycarbonyl-(L)-tert-leucine as used in all aspects of present invention can be obtained by reacting N-methoxycarbonyl-(L)-tert-leucine with a coupling agent such as O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide, N-ethyl-N′-dimethylaminopropylcarbodiimide (EDC), etc.

While the present invention has been described in terms of its specific aspects, certain modifications and equivalents will be apparent to those skilled in the art and are intended to be within the scope of the present invention.

In the following section, aspects are described by way of examples to illustrate the processes of the invention. However, these are not intended in any way to limit the scope of the present invention. Several variants of these examples would be evident to persons ordinarily skilled in the art.

### EXAMPLES

### Example 1

### Preparation of (2S,3R)-2-Amino-4-chloro-1-phenylbutan-3-ol hydrochloride

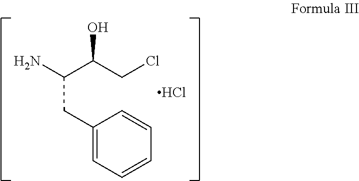

To 1,1-dimethylethyl[(2S,3R)-4-chloro-3-hydroxy-1-phenylbutan-2-yl]carbamate (Formula II, 200 g), dichloromethane (400 mL) and de-ionized water (400 mL) were added at ambient temperature under stirring. Concentrated hydrochloric acid (136 mL) was slowly added to the reaction mixture. The resultant reaction mixture was heated to 40° C. to 45° C. Dichloromethane was recovered and the reaction mixture was stirred for 2 hours to 3 hours at 50° C. to 55° C. Completion of the reaction was monitored by HPLC. After completion of the reaction, the reaction mixture was cooled to 20° C. to 25° C., dichloromethane (200 mL) was added and the mixture was stirred for 10 minutes to 15 minutes at 20° C. to 25° C. and was then allowed to stand to settle the layers. The organic layer was discarded and the aqueous layer was carried forward to the next step.

### Example 1(A)

### Preparation of (2S,3R)-2-Amino-4-chloro-1-phenylbutan-3-ol hydrochloride

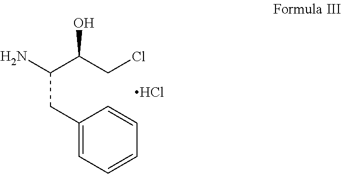

To 1,1-dimethylethyl[(2S,3R)-4-chloro-3-hydroxy-1-phenylbutan-2-yl]carbamate (Formula II, 40 g), tetrahydrofuran (200 mL) was added at ambient temperature under stirring. Concentrated hydrochloric acid (27 mL) was slowly added to the mixture. The resultant reaction mixture was heated at 50° C. to 55° C. for 4 hours. Tetrahydrofuran was recovered under vacuum from the reaction mixture at 50° C. to 55° C. Water was removed by adding additional tetrahydrofuran (200 mL) to get the semi-solid product. The semi-solid product was crystallized with a mixture of ethyl acetate (80 mL) and diisopropyl ether (200 mL) and further stirred for 2 hours at 20° C. to 25° C. The solid so obtained was filtered and dried under vacuum at 40° C. to 45° C. to afford the title compound.

Weight: 32.0 g

Yield (w/w): 0.8

### Example 2

### Preparation of Methyl[(2S)-1-{[(2S,3R)-4-Chloro-3-hydroxy-1-phenylbutan-2-yl]amino}-3,3-dimethyl-1-oxobutan-2-yl]carbamate

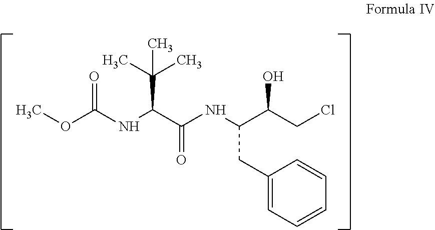

Anhydrous condition was kept during preparation method.

To N-methoxycarbonyl-(L)-tert-leucine (138.85 g), 1-hydroxybenzotriazole [HOBT, 108.16 g] was added at ambient temperature under nitrogen atmosphere. The reaction mixture was cooled to 0° C. to −5° C. and triethyl amine (168.62 g) was slowly added to it. Dichloromethane (1600 mL) was added to the reaction mixture at ambient temperature followed by the slow addition of thionyl chloride (91.28 g) at 0° C. to 5° C. within a period of 1.5 hours to 2.0 hours under nitrogen atmosphere. The resultant reaction mixture was heated to 10° C. to 15° C., stirred for 1.5 hours to 2 hours, and de-ionized water (1000 mL) was added. This was stirred for 10 minutes to 15 minutes at ambient temperature and was allowed to settle into layers. The aqueous layer was discarded and the organic layer was cooled to 5° C. to 15° C. Aqueous solution of dipotassium hydrogen orthophosphate (K₂HPO₄; 460 g/2000 mL de-ionized water) was added to the organic layer.

To the above mixture, the aqueous layer of (2S,3R)-2-amino-4-chloro-1-phenylbutan-3-ol hydrochloride (entire quantity as obtained in Example 1) was added slowly over a period of 1.5 hours to 2 hours at 5° C. to 15° C. and the resultant reaction mixture was heated to 30° C. to 35° C. for 12 hours to 14 hours. Completion of the reaction was monitored by HPLC. After completion of the reaction, the reaction mixture was cooled to 20° C. to 25° C., the aqueous solution of potassium carbonate (120 g/1000 mL de-ionized water) was added to the mixture and stirred for 10 minutes to 15 minutes at 20° C. to 25° C. The layers were separated. The aqueous layer was discarded. The aqueous solution of hydrochloric acid (200 mL diluted in 1000 mL de-ionized water) was added to the organic layer at 20° C. to 25° C. and stirred for 10 minutes to 15 minutes at ambient temperature. The organic layer was separated and washed with de-ionized water (1000 mL). The organic layer was carried forward to the next step.

### Example 2(A)

### Preparation of Methyl[(2S)-1-{[(2S,3R)-4-chloro-3-hydroxy-1-phenylbutan-2-yl]amino}-3,3-dimethyl-1-oxobutan-2-yl]carbamate

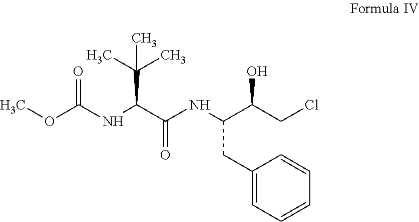

To N-methoxycarbonyl-(L)-tert-leucine (69.5 g), 1-hydroxybenzotriazole [HOBT; 54.2 g] and dichloromethane (800 mL) were added at ambient temperature under a nitrogen atmosphere. The reaction mixture was cooled to 0° C. to 10° C. and triethyl amine (84.4 g) was slowly added to it. Thionyl chloride (45.75 g) was added to the reaction mixture and it was heated to 20° C. to 25° C., stirred for 3 hours and thereafter cooled to 10° C. to 15° C. The aqueous solution of K₂HPO₄ (230 g in 500 mL de-ionized water) was added to the reaction mixture.

To the above reaction mixture, the aqueous layer of (2S,3R)-2-amino-4-chloro-1-phenylbutan-3-ol hydrochloride (as obtained in Example 1) was added slowly and the reaction mixture was heated to 30° C. to 35° C. and stirred for 12 hours. Completion of the reaction was monitored by HPLC. After completion of the reaction, an aqueous solution of sodium carbonate (42.5 g in 200 mL de-ionized water) was slowly added, further stirred for 20 minutes and the mixture was allowed to settle. Two layers were separated. The aqueous layer was discarded and the organic layer was washed sequentially with an aqueous solution of hydrochloric acid (100 mL concentrated hydrochloric acid in 300 mL de-ionized water) and de-ionized water (100 mL). The organic layer was dried to afford the title compound.

Weight: 34.0 g

Yield (w/w): 0.68

### Example 3

### Preparation of Methyl[(2S)-3,3-Dimethyl-1-({(1S)-1-[(2R)-oxiran-2-yl]-2-phenylethyl}amino)-1-oxobutan-2-yl]carbamate

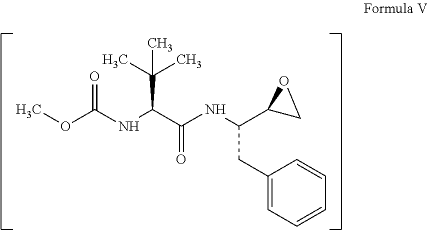

N-1-(tert-butoxycarbonyl)-N-2-[4-(pyridine-2-yl)benzyl]hydrazine (159.78 g) and tetrabutyl ammonium bromide (TBAB; 10 g) were added to the organic layer of methyl[(2S)-1-{[(2S,3R)-4-chloro-3-hydroxy-1-phenylbutan-2-yl]amino}-3,3-dimethyl-1-oxobutan-2-yl]carbamate (Formula IV; obtained in Example 2) at ambient temperature. The reaction mixture was cooled to 0° C. to 5° C. and an aqueous solution of potassium hydroxide (74.87 g/1000 mL de-ionized water) was added slowly over a period of 45 minutes to 60 minutes. The reaction mixture was stirred for 2 hours to 3 hours at 0° C. to 5° C. The completion of the reaction was monitored by HPLC. After the completion of the reaction, the organic layer was separated and washed with de-ionized water (1000 mL). The organic layer was carried forward to the next step. This layer was comprised of N-1-(tert-butoxycarbonyl)-N-2-[4-(pyridine-2-yl)benzyl]hydrazine and the title compound.

### Example 3(A)

### Preparation of Methyl 1(2S)-3,3-Dimethyl-1-({(1S)-1-1(2R)-oxiran-2-yl]-2-phenylethyl}amino)-1-oxobutan-2-yl]carbamate

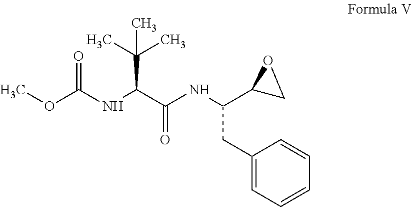

Tetrabutyl ammonium bromide (TBAB, 2 g) was added to the solution of methyl[(2S)-1-{[(2S,3R)-4-chloro-3-hydroxy-1-phenylbutan-2-yl]amino}-3,3-dimethyl-1-oxobutan-2-yl]carbamate (Formula IV; obtained in Example 2) in dichloromethane (400 mL). The reaction mixture was cooled to 0° C. to 10° C. and an aqueous solution of potassium hydroxide (18.72 g in 250 mL de-ionized water) was added slowly. The reaction mixture was stirred for 3 hours at 0° C. to 10° C. The completion of the reaction was monitored by HPLC. After the completion of the reaction, the organic layer was separated and washed twice with de-ionized water (2×100 mL). The organic layer was concentrated to dryness. To the dried mass, a mixture of acetone (225 mL) and de-ionized water (225 mL) was added. The reaction mixture was stirred for 2 hours at ambient temperature, filtered and washed with a mixture of acetone (50 mL) and de-ionized water (50 mL). The reaction mixture was dried in an air oven at 50° C. to 55° C. for a period of 12 hours to afford the title compound.

Weight: 31.0 g

Yield (w/w): 0.62

### Example 4

### Preparation of 1-[4-(Pyridine-2-yl)-phenyl]-4(s)-hydroxy-2-N-tert-butoxycarbonylamino-5(S)—N—(N-methoxycarbonyl-(L)-tert-leucyl)amino-6-phenyl-2-azahexane

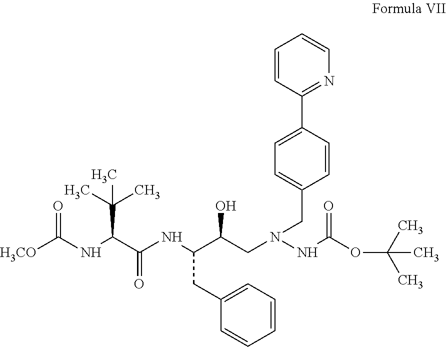 [SMILES: COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

To the organic layer of methyl[(2S)-3,3-dimethyl-1-({(1S)-1-[(2R)-oxiran-2-yl]-2-phenylethyl}amino)-1-oxobutan-2-yl]carbamate and N-1-(tert-butoxycarbonyl)-N-2-[4-(pyridine-2-yl)benzyl]hydrazine (obtained in Example 3; entire quantity), de-ionized water (2600 mL) was added and the reaction mixture was heated to 40° C. to 45° C. to recover dichloromethane atmospherically. The reaction mixture was stirred for 14 hours to 16 hours at 60° C. to 65° C. The completion of the reaction was monitored by HPLC. After the completion of the reaction, the reaction mixture was filtered and the wet cake obtained was washed with de-ionized water (400 mL) at 60° C. to 65° C. The wet cake was added to dichloromethane (1600 mL) at 20° C. to 25° C. and stirred at 35° C. to 40° C. to get a clear solution. The reaction mixture was cooled to 25° C. to 30° C. and the organic layer was separated. Toluene (1600 mL) was added to the organic layer and dichloromethane was recovered under vacuum at 40° C. to 50° C. The reaction mixture was stirred for 20 minutes to 30 minutes at 60° C. to 65° C., then cooled to 20° C. to 25° C. and stirred at the same temperature for 2 hours to 3 hours. The reaction mixture was filtered; the wet cake obtained was washed with toluene (200 mL) and dried to afford the title compound.

Yield (w/w)=1.4

### Example 5

### Preparation of 1-[4-(Pyridine-2-yl)-phenyl]-4(S)-hydroxy-2-amino-5(S)—N—(N-methoxycarbonyl-(L)-tert-leucyl)amino-6-phenyl-2-azahexane (as dihydrochloride salt)

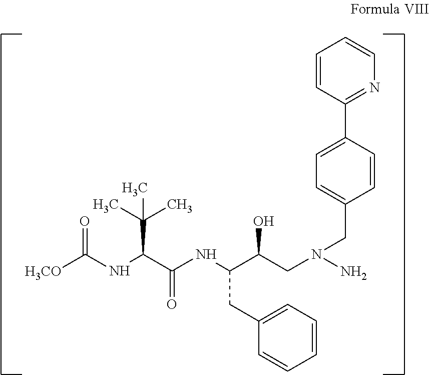

To 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII, 200 g; as obtained in Example 4), dichloromethane (400 mL) and de-ionized water (400 mL) were added under stirring at ambient temperature. Concentrated hydrochloric acid (136 mL) was slowly added to the reaction mixture over a period of 45 minutes to 60 minutes and the resultant mixture was heated to 40° C. to 45° C. to recover dichloromethane atmospherically. The reaction mixture was further stirred for 2 hours to 3 hours at 50° C. to 55° C. The completion of the reaction was monitored by HPLC. After the completion of the reaction, the reaction mixture was cooled to 20° C. to 25° C. The layers were separated. Dichloromethane (200 mL) was added to the aqueous layer with stirring for 10 minutes to 15 minutes at 20° C. to 25° C. The organic layer was discarded and the aqueous layer was carried forward to the next step.

### Example 5(A)

### Preparation of 1-[4-(Pyridine-2-yl)-phenyl]-4(S)-hydroxy-2-amino-5(S)—N—(N-methoxycarbonyl-(L)-tert-leucyl)amino-6-phenyl-2-azahexane (as dihydrochloride salt)

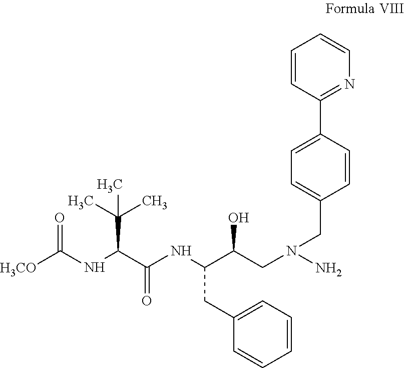 [SMILES: COC(=O)N[C@H](C(=O)N[C@@H](Cc1ccccc1)[C@@H](O)CN(N)Cc1ccc(-c2ccccn2)cc1)C(C)(C)C]

To 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII, 50 g; as obtained in Example 4), tetrahydrofuran (300 mL) was added. Concentrated hydrochloric acid (31.7 mL) was slowly added to the reaction mixture and it was stirred for 6 hours at 60° C. to 65° C. Tetrahydrofuran was recovered under vacuum from the reaction mixture. Water was removed from the reaction mixture azeotropically by adding and recovering toluene twice (2×100 mL). Ethyl acetate (100 mL) and hexane (500 mL) were added to the reaction mixture with stirring and the reaction mixture was heated to 60° C. to 65° C. The reaction mixture stood to attain the ambient temperature and was then stirred for 2 hours, filtered and washed with hexane (100 mL). The product was dried in a vacuum oven at 45° C. to 50° C. for 12 hours to afford the title compound.

Weight: 32.0 g

Yield (w/w): 0.64

### Example 6

### Preparation of Atazanavir

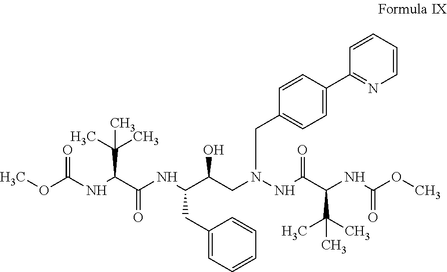

To N-methoxycarbonyl-(L)-tert-leucine (65.72 g), 1-hydroxybenzotriazole (HOBT; 51.19 g) was added at ambient temperature under nitrogen atmosphere and the reaction mixture was cooled to 0° C. to −5° C. Triethylamine (79.81 g) was slowly added to the reaction mixture at 0° C. to −5° C. and further dichloromethane (1600 mL) was added at ambient temperature. Thionyl chloride (43.20 g) was slowly added over a period of 1 hour to 1.5 hours at 0° C. to −5° C. under nitrogen atmosphere and the reaction mixture was stirred at 10° C. to 15° C. for 1.5 hours to 2 hours. De-ionized water (1000 mL) was added and the reaction mixture was stirred for 10 minutes to 15 minutes at 10° C. to 15° C. The organic layer was separated and dipotassium hydrogen orthophosphate (K₂HPO₄; 480 g/2000 mL de-ionized water) was added at 10° C. to 15° C. The aqueous layer of dihydrochloride salt of 1-[4-(pyridine-2-yl)-phenyl]-4(S)-hydroxy-2-amino-5(S)—N—(N-methoxycarbonyl-(L)-tert-leucyl)amino-6-phenyl-2-azahexane (as obtained in Example 5) was added to the organic layer over a period of 1.5 hours to 2 hours at 10° C. to 15° C. This reaction mixture was stirred for 12 hours to 14 hours at 30° C. to 35° C. The completion of the reaction was monitored by HPLC. After completion of the reaction, the reaction mixture was cooled to 20° C. to 25° C. and the organic layer was separated. The organic layer was sequentially washed with an aqueous solution of potassium hydrogen sulfate (20 g/1000 mL de-ionized water), an aqueous solution of potassium carbonate (120 g/1000 mL de-ionized water) and de-ionized water (1000 mL). The organic layer so obtained was heated to 40° C. to 45° C. to recover dichloromethane atmospherically and then vacuum was applied for 50 minutes to 60 minutes at the same temperature. Methanol (1400 mL) was added to the mass so obtained at 40° C. to 45° C. and the reaction mixture was further heated to 60° C. to 68° C. De-ionized water (1200 mL) was slowly added over a period of 30 minutes to 45 minutes at 60° C. to 68° C. The reaction mixture was heated to reflux at 72° C. to 78° C. for 30 minutes. The reaction mixture was cooled to 20° C. to 25° C. and stirred for 2 hours to 3 hours. The solid was filtered and the wet cake was washed with a mixture of methanol (200 mL) and de-ionized water (200 mL). The solid so obtained was washed with toluene (1000 mL) and dried at 60° C. to 65° C. to afford the title compound.

Yield (w/w): 1.0

### Example 7

### Preparation of Atazanavir Bisulfate (1:1)

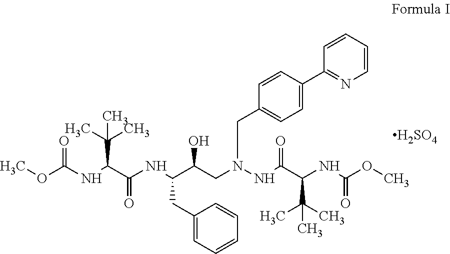

To atazanavir (150 g; obtained in Example 6), dichloromethane (900 mL) and N-methylpyrrolidine (188 mL) were added under stirring at ambient temperature and further stirred for 30 minutes to 40 minutes. After the solution became clear, activated carbon (7.5 g) was added and further stirred for 30 minutes to 40 minutes at ambient temperature.

The reaction mixture was filtered through a hyflo-bed and the bed was washed twice with dichloromethane (2×150 mL). The filtrates were combined, heated to 40° C. to 45° C. and dichloromethane was recovered atmospherically. Sequentially, acetone (2×750 mL) was added to the reaction mass, the solution so obtained was heated to 60° C. to 65° C. and acetone was recovered atmospherically and then vacuum was applied for 30 minutes to 45 minutes at 60° C. to 65° C. After that, acetone (2400 mL) was added at the same temperature and then cooled to 40° C. to 45° C. Concentrated sulfuric acid (22.96 g) was slowly added over a period of 3 hours to 4 hours at 40° C. to 45° C. and stirred for 30 minutes to 40 minutes. The reaction mixture was cooled to 20° C. to 25° C. and stirred for 3 hours to 4 hours at 20° C. to 25° C. The solid was filtered and acetone (1200 mL) was added at 20° C. to 25° C. to the wet cake and stirred for 15 minutes to 20 minutes. The solid was filtered, washed with acetone (150 mL) and dried under vacuum at 45° C. to 50° C. to afford the title compound.

Yield (w/w): 1.03

1. A process for the preparation of atazanavir or its bisulfate salt comprising the steps of:
a) converting 1,1-dimethylethyl[(2S,3R)-4-chloro-3-hydroxy-1-phenylbutan-2-yl]carbamate (Formula II) into 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII) 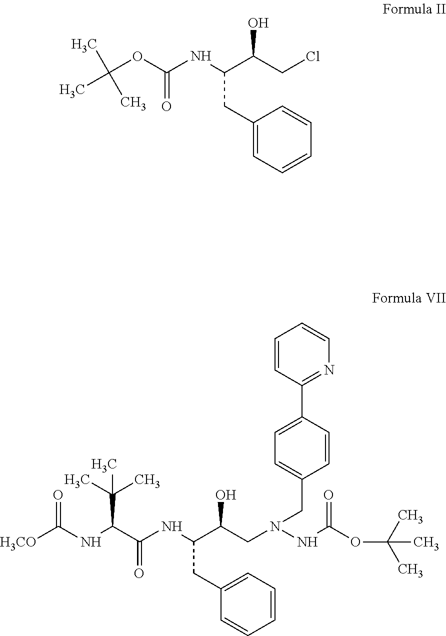 [SMILES: CC(C)(C)OC(=O)N[C@@H](Cc1ccccc1)[C@@H](O)CCl ; COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]
without isolating any intermediate compound involved in the conversion; and
b) converting the compound of Formula VII of step a) into atazanavir or its bisulfate salt.

2. The process of claim 1, wherein the compound of Formula II is converted into a compound of Formula VII without isolating intermediate compounds (2S,3R)-2-amino-4-chloro-1-phenylbutan-3-ol (Formula III), methyl[(2S)-1-{[(2S,3R)-4-chloro-3-hydroxy-1-phenylbutan-2-yl]amino}-3,3-dimethyl-1-oxobutan-2-yl]carbamate (Formula IV), and/or methyl[(2S)-3,3-dimethyl-1-({(1S)-1-[(2R)-oxiran-2-yl]-2-phenylethyl}amino)-1-oxobutan-2-yl]carbamate (Formula V).

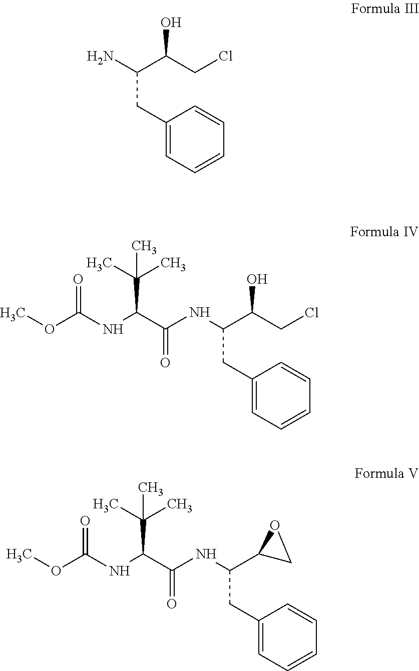

3. The process of claim 1, wherein the compound of Formula VII is converted into atazanavir or its bisulfate salt comprising the step of deprotecting a compound of Formula VII to get a compound of Formula VIII, or a salt thereof.

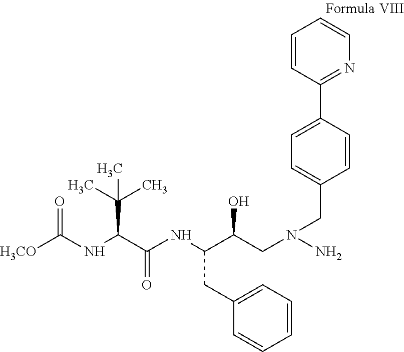 [SMILES: COC(=O)N[C@H](C(=O)N[C@@H](Cc1ccccc1)[C@@H](O)CN(N)Cc1ccc(-c2ccccn2)cc1)C(C)(C)C]

4. The process of claim 3, wherein the compound of Formula VIII or a salt thereof is further treated with an active ester of N-methoxycarbonyl-(L)-tert-leucine to obtain atazanavir or its bisulfate salt.

5. The process of claim 4, wherein the active ester is obtained by the treatment of N-methoxycarbonyl-(L)-tert-leucine with a compound selected from O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide or N-ethyl-N′-dimethylaminopropylcarbodiimide (EDC).

6. A process for preparation of atazanavir or its bisulfate salt comprising:
a) converting a compound of Formula II into a compound of Formula VII 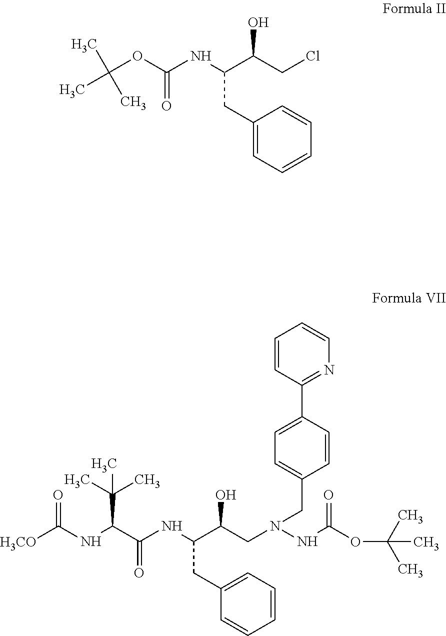 [SMILES: CC(C)(C)OC(=O)N[C@@H](Cc1ccccc1)[C@@H](O)CCl ; COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]
without isolating any intermediate from among (2S,3R)-2-amino-4-chloro-1-phenylbutan-3-ol (Formula III), methyl[(2S)-1-{[(2S,3R)-4-chloro-3-hydroxy-1-phenylbutan-2-yl]amino}-3,3-dimethyl-1-oxobutan-2-yl]carbamate (Formula IV), or methyl[(2S)-3,3-dimethyl-1-({(1S)-1-[(2R)-oxiran-2-yl]-2-phenylethyl}amino)-1-oxobutan-2-yl]carbamate (Formula V); and 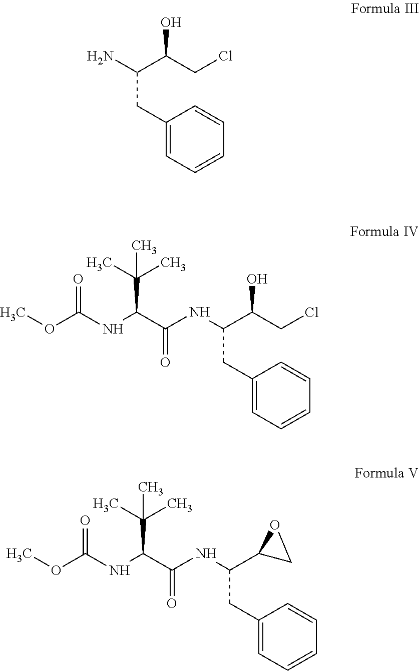
b) converting the compound of Formula VII of step a) into atazanavir or its bisulfate salt.

7. The process of claim 6, wherein the compound of Formula VII is converted into atazanavir or its bisulfate salt using a process comprising the step of deprotecting a compound of Formula VII to get a compound of Formula VIII, or a salt thereof.

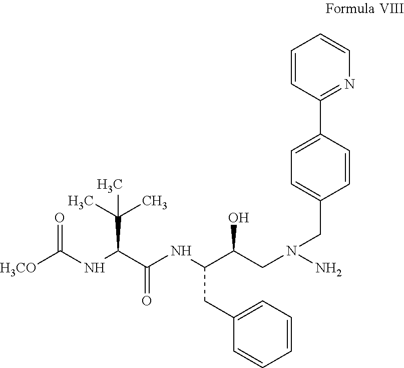 [SMILES: COC(=O)N[C@H](C(=O)N[C@@H](Cc1ccccc1)[C@@H](O)CN(N)Cc1ccc(-c2ccccn2)cc1)C(C)(C)C]

8. The process of claim 7, wherein the compound of Formula VIII or a salt thereof is further treated with an active ester of N-methoxycarbonyl-(L)-tert-leucine to obtain atazanavir or its bisulfate salt.

9. The process of claim 8, wherein the active ester is obtained by the treatment of N-methoxycarbonyl-(L)-tert-leucine with a compound selected from O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide, or N-ethyl-N′-dimethylaminopropylcarbodiimide (EDC).

10. A process for the preparation of atazanavir or its bisulfate salt by treating a reaction mixture comprising the compound of Formula VIII 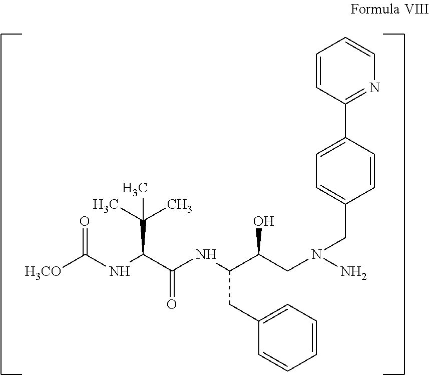
with an active ester of N-methoxycarbonyl-(L)-tert-leucine.

11. The process of claim 10, wherein the treatment of the reaction mixture with an active ester of N-methoxycarbonyl-(L)-tert-leucine is performed in the presence of thionyl chloride or oxalyl chloride.

12. The process of claim 10, wherein the reaction mixture is treated with an active ester of N-methoxycarbonyl-(L)-tert-leucine in the presence of a base and an organic solvent.

13. The process of claim 12, wherein the base is selected from sodium hydroxide, potassium hydroxide, magnesium hydroxide, dipotassium hydrogen orthophosphate, magnesium carbonate, sodium carbonate, potassium carbonate, pyridine, trimethylamine, triethylamine, diisopropylethylamine or N-methyl morpholine.

14. The process of claim 12, wherein the organic solvent is selected from methylene chloride, ethyl acetate, butyl acetate, dichloroethane, tetrahydrofuran, acetonitrile, acetone, dimethylsulfoxide, N,N-dimethylformamide, benzene, toluene, 1,4-dioxane, chloroform or diethyl ether.

15. The process of claim 10, wherein the active ester is obtained by the treatment of N-methoxycarbonyl-(L)-tert-leucine with a compound selected from O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide or N-ethyl-N′-dimethylaminopropylcarbodiimide (EDC).

16. A process for the preparation of atazanavir or its bisulfate salt comprising a step of reacting (2S,3R)-2-amino-4-chloro-1-phenylbutan-3-ol of Formula III or a salt thereof 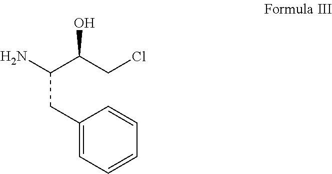
with an active ester of N-methoxycarbonyl-(L)-tert-leucine in presence of thionyl chloride or oxalyl chloride to get methyl[(2S)-1-{[(2S,3R)-4-chloro-3-hydroxy-1-phenylbutan-2-yl]amino}-3,3-dimethyl-1-oxobutan-2-yl]carbamate of Formula IV.

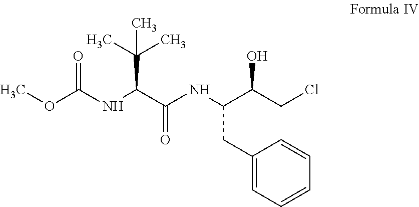

17. The process of claim 16, wherein the reaction of the compound of Formula III or a salt thereof with an active ester of N-methoxycarbonyl-(L)-tert-leucine is performed in the presence of a base and an organic solvent.

18. The process of claim 17, wherein the base is selected from sodium hydroxide, potassium hydroxide, magnesium hydroxide, dipotassium hydrogen orthophosphate, magnesium carbonate, sodium carbonate, potassium carbonate, pyridine, trimethylamine, triethylamine, diisopropylethylamine or N-methyl morpholine.

19. The process of claim 17, wherein the organic solvent is selected from methylene chloride, ethyl acetate, butyl acetate, dichloroethane, tetrahydrofuran, acetonitrile, acetone, dimethylsulfoxide, N,N-dimethylformamide, benzene, toluene, 1,4-dioxane, chloroform or diethyl ether.

20. The process of claim 16, wherein the active ester is obtained by the treatment of N-methoxycarbonyl-(L)-tert-leucine with a compound selected from O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide, or N-ethyl-N′-dimethylaminopropylcarbodiimide (EDC).

21. A process for the preparation of atazanavir or its bisulfate salt comprising a step of condensing in-situ methyl[(2S)-3,3-dimethyl-1-({(1S)-1-[(2R)-oxiran-2-yl]-2-phenylethyl}amino)-1-oxobutan-2-yl]carbamate (Formula V) 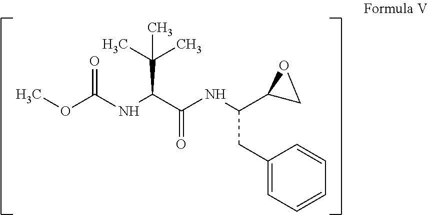
with N-1-(tert-butoxycarbonyl)-N-2-[4-(pyridine-2-yl)benzyl]hydrazine of Formula VI 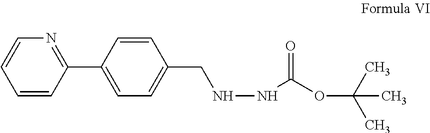 [SMILES: CC(C)(C)OC(=O)NNCc1ccc(-c2ccccn2)cc1]
to obtain 1-[4-(pyridine-2-yl)-phenyl]-4(S)-hydroxy-2-N-tert-butoxycarbonylamino-5(S)—N—(N-methoxycarbonyl-(L)-tert-leucyl)amino-6-phenyl-2-azahexane (Formula VII).

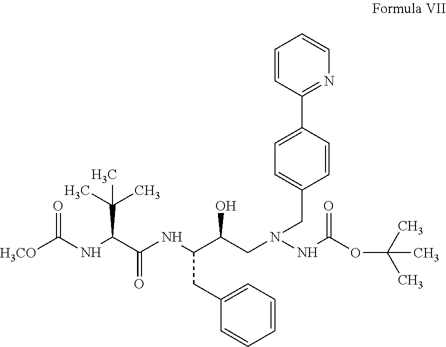 [SMILES: COC(=O)N[C@H](C(=O)N[C@@H](Cc1ccccc1)[C@@H](O)CN(Cc1ccc(-c2ccccn2)cc1)NC(=O)OC(C)(C)C)C(C)(C)C]

22. The process of claim 21, wherein the compound of Formula VII is further converted into atazanavir or its bisulfate salt using a process comprising the step of deprotecting a compound of Formula VII to get a compound of Formula VIII or a salt thereof.

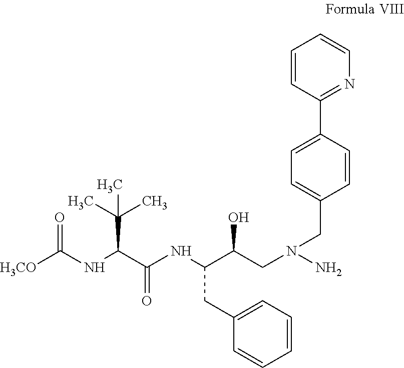 [SMILES: COC(=O)N[C@H](C(=O)N[C@@H](Cc1ccccc1)[C@@H](O)CN(N)Cc1ccc(-c2ccccn2)cc1)C(C)(C)C]

23. The process of claim 22, wherein the compound of Formula VIII or a salt thereof is further treated with an active ester of N-methoxycarbonyl-(L)-tert-leucine to obtain atazanavir or its bisulfate salt.

24. The process of claim 23, wherein the active ester is obtained by the treatment of N-methoxycarbonyl-(L)-tert-leucine with a compound selected from O-(1,2-dihydro-2-oxo-1-pyridyl)-N,N,N¹,N¹-tetramethyluronium tetrafluoroborate (TPTU), 1-hydroxybenzotriazole (HOBT), N-hydroxysuccinamide, or N-ethyl-N′-dimethylaminopropylcarbodiimide (EDC).
